(19) United States
(12) Patent Application Publication
(10) Pub. No.: US 2021/0291167 A1
(43) Pub. Date: Sep. 23, 2021

(54) STRIATED TEST TUBE AND METHOD OF FLUID TRANSFER USING THE SAME

(71) Applicant: Roche Diagnostics Hematology, Inc., Westborough, MA (US)

(72) Inventors: Christopher Donat, Boston, MA (US); Daniel Lapen, Lancaster, MA (US); Bernard Lane, Littleton, MA (US); Stephen Conroy, Maynard, MA (US)

(21) Appl. No.: 17/186,597

(22) Filed: Feb. 26, 2021

Related U.S. Application Data
(63) Continuation of application No. PCT/US2019/048535, filed on Aug. 28, 2019.
(60) Provisional application No. 62/723,791, filed on Aug. 28, 2018.

(51) Int. Cl. B01L 3/00

(52) U.S. Cl. CPC B01L 3/50825; B01L 13/02; B01L 2300/042; B01L 2400/049; B01L 2300/0858; B01L 2300/165; B01L 2300/0832

(57) ABSTRACT

A fluid-holding vessel (28) with a surface tension reducing geometry which comprises an inner surface having striations (24) and a fluid transfer method are disclosed and described. The fluid-holding vessel (28) may be a test tube that is used in combination with a cap (20), which is penetrable by a fluid transfer device (11) of an automated analyzer (10) used to transfer fluids to or from the striated test tube, where the tube and cap may remain physically and sealably associated during a fluid transfer. The automated analyzer (10) may be used in combination with the fluid-holding vessel (28) as disclosed and described herein, in which the surface tension reducing geometry (24, 26) of the vessel (28) addresses an aspiration problem of a liquid (18) dispensed therefrom automatically by the automated analyzer (10), e.g., into a sample cup.

### CROSS-REFERENCE TO RELATED APPLICATIONS

This application is a continuation, filed under 35 U.S.C. 111(a), of International Patent Application No. PCT/US2019/048535, filed Aug. 28, 2019, which claims priority to U.S. Provisional Patent Application No. 62/723,791 filed Aug. 28, 2018, the entire disclosures of which are hereby incorporated by reference.

### TECHNICAL FIELD

This application is directed generally to fluid-holding vessels, and in particular to a fluid-holding vessel such as, for example, in the form of a test tube, with a surface tension reducing inner surface striated geometry which addresses aspiration problems of cleaning fluids or liquids dispensed automatically by an automated analyzer, e.g. into a sample cup. This application is also directed to the fluid-holding vessel in combination with a cap, the fluid-holding vessel in combination with an automated analyzer, and a fluid transfer method using the fluid-holding vessel.

### BACKGROUND

A quantitative, automated analyzer is a laboratory instrument designed to measure different chemicals and other characteristics in a number of biological samples quickly, with minimal human assistance. Generally, such an analyzer consists of the following major components: the analyzer, with rack transport system for sample test tubes; viewing stations that can be configured as the control station or as a review station; and associated consumables and components. Typically, one of the associated consumables is a cleaning solution that is provided in a test tube and used automatically in such an analyzer to remove contaminants, such as protein build-up, from the surfaces of the analyzer components that come in contact with the biological sample(s). Some analyzers require the biological samples be transferred to sample cups before analysis, which thus need to be cleaned after each use. Some analyzers aspirate automatically the cleaning fluid from the provided test tube and apply it to the sample cup during a cleaning cycle.

### SUMMARY

It is against that above background that in one generalized embodiment, a fluid-holding vessel with a surface tension reducing inner surface striated geometry which addresses an aspiration problem of a cleaning fluid or liquid dispensed automatically by an automated analyzer, e.g. into a sample cup is disclosed.

In another generalized embodiment, the fluid-holding vessel may be in the form of a striated test tube that is for use in combination with a cap, which is penetrable by a fluid transfer device of an automated analyzer used to transfer fluids to or from the striated test tube, where the tube and cap remain physically and sealably associated during a fluid transfer.

In still another generalized embodiment, disclosed is an automated analyzer in combination with a fluid-holding vessel with a surface tension reducing inner surface striated geometry, said striated geometry of the vessel being configured to address an aspiration problem of a cleaning fluid or liquid dispensed therefrom automatically by the automated analyzer, e.g. into a sample cup.

In still yet another generalized embodiment, disclosed is a fluid transfer method in which a fluid is drawn from a fluid-holding vessel with a surface tension reducing inner surface striated geometry via a fluid transfer device of an automated analyzer penetrating a cap physically and sealably associated with the vessel during a fluid transfer, where the surface tension reducing inner surface striated geometry of the vessel addresses aspiration problems of a cleaning fluid or liquid contained therein and dispensed by the fluid transfer device, e.g. into a sample cup.

These and other features, aspects, and advantages of the various embodiments discussed herein will become apparent to those skilled in the art after considering the following detailed description, appended claims and accompanying drawings.

### BRIEF DESCRIPTION OF THE DRAWINGS

FIG. 1 is a depiction of a portion of an automated analyzer holding a conventional test tube in an inverted orientation for aspirating a liquid therefrom;

FIG. 2 is a cross-section view of a portion of a fluid-holding vessel detailing a striated design according to an embodiment of the present invention which addresses a dispensing problem of a cleaning fluid or liquid contained therein;

FIG. 3 is a section view detailing a conventional test tube shown in cross-section which has a dispensing problem with a cleaning fluid or liquid contained therein;

FIG. 4 is a cross-section view of a concave striated vessel according to an embodiment of the present invention;

FIG. 4A is a section view of the concave striated vessel taken at section line 4A-4A in FIG. 4;

FIG. 5 is a cross-section view of a convex striated vessel according to an embodiment of the present invention;

FIG. 5A is a section view of the convex striated vessel taken at section line 5A-5A in FIG. 5; and

FIG. 6 is a depiction of a portion of an automated analyzer holding a striated vessel according to an embodiment of the present invention in an inverted orientation for aspirating a liquid therefrom.

### DETAILED DESCRIPTION

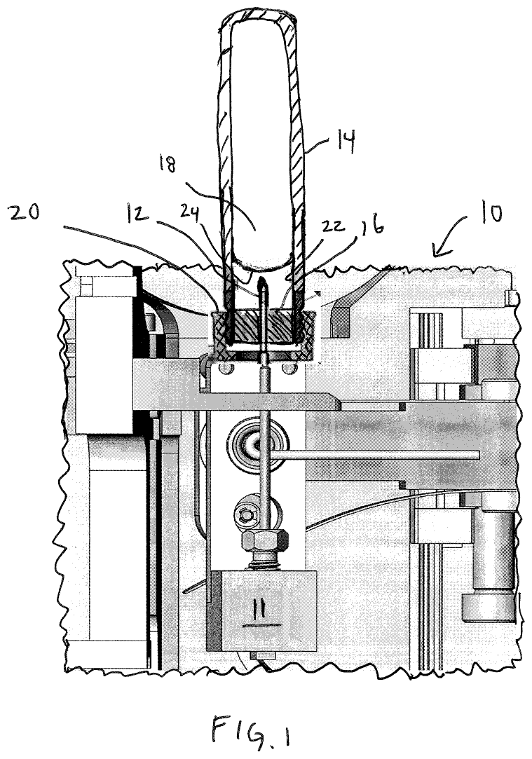

Referring to FIG. 1, depicted generally is an automatic analyzer 10, and in particular a fluid delivery device 11 which is a component thereof. The fluid delivery device 11 is depicted with having a piercing needle 12. As a consumable, a conventional test tube 14 (depicted in cross-section) is shown inverted and held securely in such an orientation by the analyzer 10 such that the piercing needle or probe 12 of the fluid delivery device 11 may be advanced automatically to pierce a septum 16 of the test tube 14. The inventors have discovered that with such conventional test tubes, such as the one depicted by test tube 14 which are used by automated analyzers in a fashion similar to the orientation shown by analyzer 10, can cause such analyzers to not consistently aspirate/dispense completely a liquid, a cleaning fluid or disinfectant solution 18 from the test tube 14 into, e.g. a sample cup (not shown) during a clean cycle.

For a better perspective of this discovered problem, it is to be appreciated that one type of automated analyzer which orientates the test tube 14, which is filled with a liquid or disinfectant (cleaning) solution 18 in the manner depicted by FIG. 1, is the cobas m 511 integrated hematology analyzer (Roche Diagnostics). The cobas m 511 analyzer prepares a stained microscope slide from EDTA-anticoagulated whole blood drawn from a sample cup, and then utilizes computer imaging to count the formed elements of blood and provide an image-based assessment of cell morphology. To prevent/remove protein build-up from the surfaces of analyzer components that come into contact with the blood samples, such an analyzer, i.e., analyzer 10, performs the clean cycle of the sample cup with the liquid or disinfectant solution 18, which if a disinfectant solution is typically a sodium hypochlorite based disinfectant (cleaning) solution. One example of a sodium hypochlorite based disinfectant solution noted by the inventors as not consistently being aspirate/dispense completely during such a clean cycle is the DigiMAC3™ clean solution, which is primarily a 0.7% sodium hypochlorite formulation.

Automated analyzers, like the Roche cobas m 511 analyzer, as mentioned above perform the aspirate/dispense cycle by inverting the test tube 14 containing the liquid or disinfectant solution 18 and holding it securely in the orientation depicted by FIG. 1. Thereafter, the fluid transfer device 11 pierces the needle 12 through the septum 16 and into the test tube 14, and aspirates the liquid or disinfectant solution 18 for dispensing into a sample cup (not shown). However, the particular problem discovered by the inventors and illustrated by FIG. 1, is that when the test tube 14 is inverted, the liquid or disinfectant solution 18 does not always flow to the bottom of the end of the tube with a cap 20, where the septum 16 is located. For example, it was observed/discovered that a large proportion of prior art tubes when filled with a disinfectant solution like the DigiMAC3™ clean solution, when the tube 14 was inverted with the cap 20 removed, such a disinfectant solution 18 would not flow from the tube 14.

With the aid of illustration and reference still to FIG. 1, the cause of the above noted problem has been determined to be that the surface forces of the liquid or disinfectant solution 18 to a smooth inner surface 22 of the test tube 14 are larger than the opposing gravitational force, thereby causing the liquid or disinfectant solution 18 in the test tube 14 to remain stationary when the test tube 14 is inverted. In a particular observation, a meniscus 24 caused by surface tension formed in the liquid or solution 18 above and out of reach of the needle 12 when the test tube 14 was inverted, thereby preventing the liquid or solution 18 from being aspirated from the test tube 14 by the needle 12. Although the above noted properties and characteristics depend on the composition of the liquid or disinfectant solution 18, this phenomenon noted in regards to a hypochlorite based formulation like the DigiMAC3™ clean solution can, and does, affect system performance of such analyzers, like analyzer 10, by rendering clean cycles impotent, and thereby causing the piercing/aspiration needle 12 along with the sample cup both becoming clogged and/or suffering from protein build-up, which can be particularly problematic for a pierce needle spacing algorithm employed by such analyzers. Accordingly, after discovering the above noted problem, the inventors recognized a need for a vessel, such as the various inventive vessel embodiments discussed hereinafter, that allows for liquid contents, such as a hypochlorite based liquid, to freely flow under gravitational forces, so that the piercing needle 12 of the analyzer 10 is able to aspirate the liquid or disinfectant solution 18 from within the vessel when the vessel is oriented cap-end down such as depicted by FIG. 1.

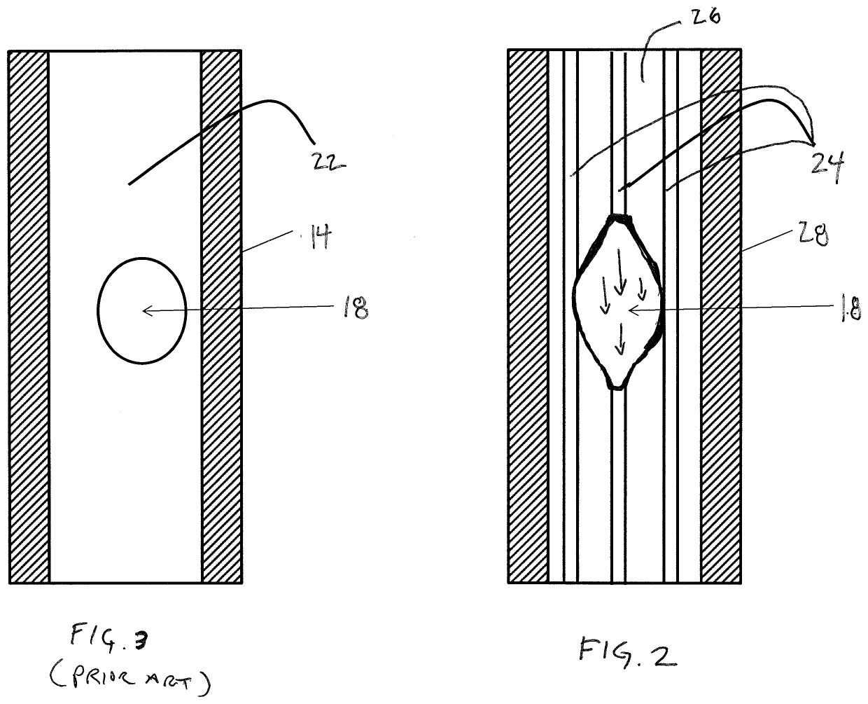

As depicted by FIG. 2, the resulting inventive solution to the above note problem is fulfilled via the addition of longitudinally extending striations 24 to the interior surface 26 of an inventive fluid-holding vessel 28. Generally, each striation 24 has a macroscopic profile (either proud or recessed) that sweeps along, e.g., the inner diameter of an example tube of vessel 28 that lies on a plane coincident and parallel to the tube's axis of revolution. These striations 24 aid in breaking surface tension, lowering the surface forces between the liquid 18, such as a hypochlorite based liquid, and the interior surface 26 of the inventive fluid-holding vessel 28. Flow from the vessel 28 of liquid contents such as water and other aqueous fluids as discussed hereinafter in the Testing & Result section in the same fashion is likewise improved by striations 24.

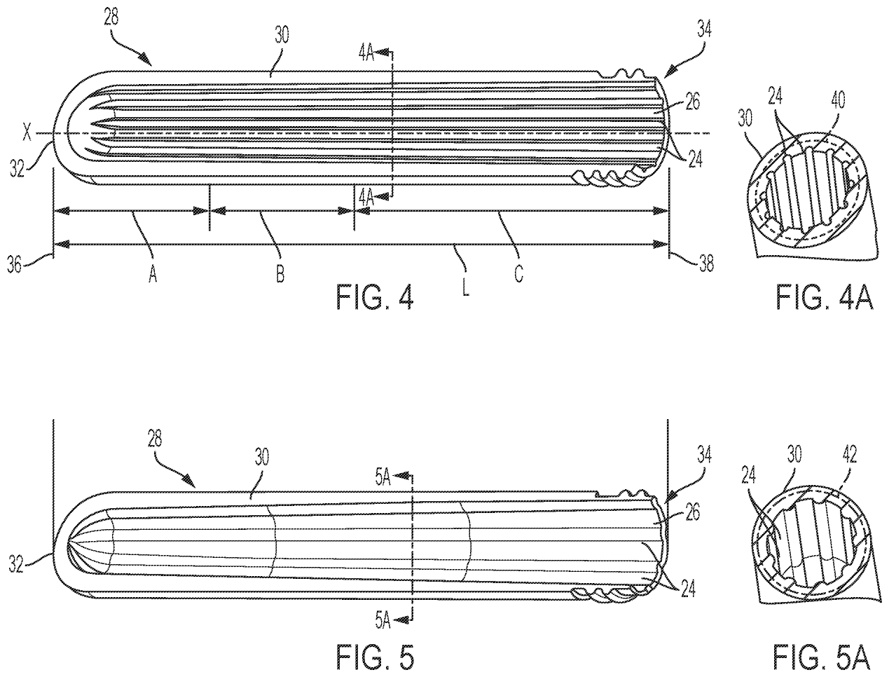

In an illustrated comparison, the shape of a drop of the liquid 18 changes from a substantially rounded symmetric shape, as depicted in FIG. 3 clinging to the smooth inner surface 22 of test tube 14, to a more elongated, flatten elliptical (oval) shape that does not cling to the interior surface 26 of the vessel 28, but rather flows easily under the force of gravity (depicted by the downwardly pointing arrows). It is to be appreciated that the longitudinal striations 24, which may be either convex or concave, are provided along the interior inner diameter (ID) parallel to a longitudinal axis (FIG. 4) of the fluid-holding vessel 28 to reduce surface tension of the interior surface 26, which addresses aspiration problems of such fluids, like liquid or disinfectant (cleaning) solution 18, that are dispensed by a fluid delivery device of automated analyzers, like device 11 of analyzer 10, into sample cups (not shown).

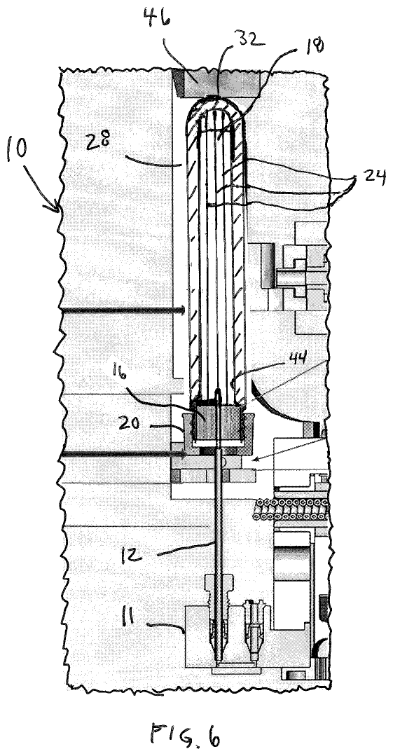

As depicted in cross-section by FIGS. 4 and 5, two different embodiments of the fluid-holding vessel 28 that may be filled with the liquid or disinfectant solution 18 such as e.g., a hypochlorite based disinfectant solution, is each shown as a cylindrical tube having a sidewall 30 and a bottom 32. However, it is understood that the vessel 28 may be any suitable shape (e.g., square, round, or triangular tubing, wells, or other containers) and may have a greater or fewer number of sidewalls (for example a square container could have four orthogonal sidewalls). While from the bottom 32 to an opposed opening 34, the sidewall 30 is illustrated as being tapered down, it is understood that in some examples at least a portion of each sidewall 30 may be straight, curved or otherwise shaped. The sidewall 30 further comprises the interior (major) surface 26 for contacting the solution 18 (FIG. 2) retained in the vessel 28.

In one embodiment, each sidewall 30 of the vessel 28 may have a continuous taper (draft of the inner ID), e.g., ranging from 1° to 3°, and preferably 2° in another embodiment. In still other embodiments, each sidewall 30 may have a varying taper (draft) along length L of the vessel 28. For example, as depicted by FIG. 4, in one embodiment a first taper, e.g., 0.5°, for a first portion A from the bottom 32, a second portion B with, e.g., a 1° of taper, and a third portion C comprising the remainder of the length L of the vessel 28 to the opening 34 provided with, e.g., 2° of taper. In an embodiment, the first portion A ranges in length from 0.5 to 1.5 inches (1.27 cm to 3.81 cm) from the bottom 32, the second portion B from 0.5 to 1.5 inches (1.27 cm to 3.81 cm), and in a preferred embodiment portions A and B are each 1 inch (2.54 cm) in length.

In the illustrated embodiments of FIGS. 4 and 5, the vessel bottom 32 is curved, while in other examples the vessel bottom 32 may be flat, sloped, concave, convex or any other suitable shape. Regardless of the actual shape of the vessel bottom 32, the vessel 28 (as depicted in FIG. 4) defines a first plane 36 adjacent the bottom 32 that is spaced apart from a second plane 38 intersecting the opening 34 of the vessel 28 to define the longitudinal length L between the first and second planes 36, 38. In addition to the longitudinal length L, a central axis X of the vessel 28 is defined between the planes 36, 38.

As also depicted in the illustrated examples of FIGS. 4 and 5, a plurality of the striations 24 runs in-plane with the central axis X of the vessel 28. Each striation 24 may be concave (best shown by FIG. 4A) or convex (best shown by FIG. 5A). The number of striations 24 may range from 4 to 24, and more preferably 8 to 12 in other embodiments, wherein the lesser number of striations and/or the type of shape, i.e., convex versus concave, may be based and preferred if a simpler core design for an injection mold tool for the vessel 28 dimensions is a desire. Convex striations also may be used and preferred in applications were maintaining a minimum wall thickness to the test tube is a desire. The striations may be spaced equally from each other, e.g., measured valley-to-valley in the case of concave striations or top-to-top in the case of convex striations, or spaced unequally from each other. Each striation 24 may also have the same shape as the other striations or can be different therefrom. For example, vessel 28 may have alternating patterns of striations 24 of different shapes, e.g., wider and/or narrow valleys in the case of concave striations, and/or higher or short hills in the case of convex striations. The vessel 28 may also be provided with both concave and convex striations, also in alternative patterns. As also indicated by the dashed lines 40 and 42, the sidewall 30 may have a thickness indicated inside each dashed line such that the striations 24 are provided as part of an insert. Such an insert can then be used to convert a conventional tube (whose sidewall thickness would be the material indicated outside of the dashed lines 40 and 42) to the inventive vessel 28. The vessel 28 may be constructed from any material that is suitable for the introduction of the striations 24. Examples of suitable materials include polymeric materials, polystyrene, polypropylene, polycarbonate, polyvinylchloride, polytetra-fluoroethylene, or other suitable polyolefin.

In one particular embodiment, vessel 28 is a solid cylindrical tube made of polypropylene, has a length L ranging from 7 to 8 cm, an outside diameter of 1 to 2 cm, provided with threads meeting the GCMI/SPI 13-425 thread specification, and an internal draft that ranges from 0.4 to 0.6 degrees. On the interior of this particular embodiment, the vessel 28 has 12 concave striations space equally every 30 degrees, measured valley-to-valley. A cross section of each striation is the same (identical) to each other and which remains normal to the path of the striation, and has a depth that ranges from 0.5 to 0.6 mm below the (major) interior surface 26 of the sidewall 30, with a minor radius that ranges from 0.3 to 0.4 mm, and a major radius that ranges from 3 to 4 mm. The minor internal diameter that is adjacent the bottom 32 of this particular embodiment ranges from 0.7 to 0.8 cm, and the major internal diameter that is adjacent the opening 34 ranges from 0.8 to 0.9 cm.

It is to be appreciated that the illustrated embodiments are designed to be injection molded and therefore are provided with a suitable draft such that the vessel 28 may be removed easily from a mold. The fluid-holding vessel 28 may have a similar major internal diameter (ID) and/or threading to conventional test tubes, like test tube 14 and those listed in Table 1, but not limited thereto.

TABLE 1
Relationship of Test tube size, threads and septum diameter

| | | | Cap Thread | | |
|---|---|---|---|---|---|
| Volume | | Vial O.D. × Height | (GCMI Spec.) | Septum Diameter | |
| 2 | mL (0.5 drams) | 12 × 32 mm × 4.6 (ID) mm | 8-425 | 8 | mm |
| 2 | mL (0.5 drams) | 12 × 32 mm × 6.0 (ID) mm | 9 mm | 9 | mm |
| 2 | mL (0.5 drams) | 12 × 32 mm × 6.0 (ID) mm | 10-425 | 10 | mm |
| 4 | mL (1 dram) | 15 × 45 mm | 13-425 | 11 | mm |
| 7 | mL (2 drams) | 17 × 60 mm | 15-425 | 13 | mm |
| 15 | mL (4 drams) | 21 × 70 mm | 18-400 | 16 | mm |
| 22 | mL (6 drams) | 23 × 85 mm | 20-400 | 18 | mm |
| 40 | mL (10.7 drams) | 29 × 81 mm | 24-400 | 22 | mm |
| 40 | mL (10.7 drams) | 28 × 98 mm | 24-400 | 22 | mm |

In the embodiment depicted by FIG. 6, the vessel 28 is a striated test tube used in combination with cap 20 in the automated analyzer 10 as a consumable which contains the liquid 18. In use, the vessel 28 and cap 20 remain physically and sealably associated during a fluid transfer. In one embodiment, the cap 20 is a polypropylene cap with a PTFE/silicone seal which demonstrates good material compatibility to the sodium hypochlorite based solutions and mechanical response to multiple piercings by the pierce needle 12. Other conventional caps may also be used. In another embodiment, a septum 16 can be provided to the vessel 28 which is penetrable by the piercing needle 12 of the fluid transfer device 11 to transfer fluids to or from the vessel 28, if the cap 20 provides no such penetrable seal.

In use, the automated analyzer 10 performs an aspirate/dispense cycle for cleaning by inverting the vessel 28 and holding it securely in the orientation depicted by FIG. 6, i.e., the bottom 32 being above the cap 20. Thereafter, the fluid transfer device 11 pierces the needle 12 through the septum 16 and into the vessel 28, and aspirates the liquid or disinfectant solution 18 for dispensing into, e.g. a sample cup (not shown). However, unlike with the conventional test tube 14 depicted in FIG. 1, the surface tension of the liquid or solution 18 is reduced enough by the striations 24 such that no aspiration problems occur by the cleaning fluid resisting flow towards the end of the vessel 28 adjacent the needle 12, i.e., does not cling to surface 26. Accordingly, the needle 12 properly functions as designed to draw the liquid or solution 18 from the vessel 28 such that the fluid transfer device 11 may dispense the liquid solution 18 into a sample cup.

It is to be appreciated that the embodiments disclosed herein are ones that do not require software, hardware, or formulation changes to the analyzer and/or disinfectant solution. However, in combination with the herein disclosed interior geometry changes to the fluid vessel 28 that is filled with the liquid or disinfectant solution 18, such as the DigMAC3™ clean solution, a material that is different from at least the sidewall 30 (FIG. 4) forming material and provided with a higher surface energy could also be employed that comes into contact with the liquid or solution 18. For example, the interior surface 44 (FIG. 6) of the vessel 28 could be fluorinated by fluorosealing, which can increase the surface energy of various plastics as shown in Table 2 (listed in units of mN/m). For example, for polypropylene, fluorosealing ups the surface energy to 70 mN/m from a base of 29 mN/m.

TABLE 2
Change in surface energy via surface fluorination

| | untreated | fluorinated |
|---|---|---|
| PE | 32 | 70 |
| PP | 29 | 70 |
| POM | 40 | 72 |
| PET | 32 | 72 |
| PBT | 30 | 72 |
| PC | 32 | 70 |
| EPDM | 40 | 58 |
| Water | 70 | |

### Testing & Results

A regression analysis was performed, resulting in the execution of two verification protocols on the inventive vessel 28. These include an aspirate and dispense test, which passed with 100% of aspiration/dispense cycles (10 tubes, 40 test cycles per tube). A pour test was also performed which showed that the striated design of the vessel 28 can assure that fluid will flow out of an inverted tube 100% of the time. An additional test that was performed was a leakage test. In this leakage test the cap 20 was able to properly seal in the contents of the tube while subjected to a −12 psi vacuum environment for a period of time greater than 12 hours, No leaks of liquid were observed. The inventive vessel 28 was compared against a conventional 13 mm test tube, which is a relatively standard size. Both the conventional 13 mm test tube and the inventive vessel 28 were used in combination with a Chemglass CG-4910-15 cap providing an SPI 13-425 standard thread.

### A. Uncap & Tip Over Testing

This testing required uncapping tubes, ensuring they had the desired liquid and volume, and inverting them using a tube gripper 46 (FIG. 6) of the analyzer 10 to quickly demonstrate whether the inventive vessel 28 (hereinafter referred as “striated tubes 28”) performed better than the conventional 13 mm test tubes. For this test, a disinfectant solution (DI) of household bleach and water was used to approximate the DigiMAC3™ clean solution (hereinafter referred to as “Clean”). Fifteen conventional 13 mm test tubes and twenty-five inventive striated tubes 28 were used in this test. Per Table 3, some striated tubes 28 were used only once while others were rinsed and refilled (due to scarcity of the striated tubes 28). Tubes highlighted came from the same lot.

The results of this testing reveal three things:

a. A simulated bleach solution (D1) is not a good representation of how actual DigiMAC3™ clean solution behaves (Test 1);
b. Water performs worse than Clean (it sticks better to a tube's interior surface). Thus, a conservative method of testing can use water instead of Clean; and
c. The striated tubes, when comparing apples to apples (Test 3), fix the problem, even allowing what would normally be dead volume to flow from tube (Test 4).

TABLE 3
Tube tip over testing results

| Tube Tip Over | | | | | | | | | | | | | | | | | | | | | | | | | |
|---|---|---|---|---|---|---|---|---|---|---|---|---|---|---|---|---|---|---|---|---|---|---|---|---|---|
| Testing Resuits | Protomold | | | | | | | | | | | | | | | | | | | | | | | | |
| Tube # | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 |
| Test 1 | | | | | | | | | | | | | | | | | | | | | | | | | |
| Tubes filled with | 1 | 1 | 1 | 1 | 1 | 1 | 1 | 1 | 1 | 1 | 1 | 1 | 1 | 1 | 1 | 1 | 1 | 1 | 1 | 1 | — | — | — | — | — |
| RDH formulated | | | | | | | | | | | | | | | | | | | | | | | | | |
| Bleach Solution | | | | | | | | | | | | | | | | | | | | | | | | | |
| and inverted on | | | | | | | | | | | | | | | | | | | | | | | | | |
| instrument | | | | | | | | | | | | | | | | | | | | | | | | | |
| Test 2 | | | | | | | | | | | | | | | | | | | | | | | | | |
| Tubes filled at RTD | — | — | — | — | — | — | — | — | — | — | — | — | — | — | — | — | — | — | — | — | — | — | — | — | — |
| Test3 | | | | | | | | | | | | | | | | | | | | | Filled with 3.5 mL DI | | | | |
| Tubes filled with DI | — | — | — | — | — | — | — | — | — | — | — | — | — | — | — | — | — | — | — | — | 1 | 1 | 1 | 1 | 1 |
| Test 4 | | | | | | | | | | | | | | | | | | | | | Filled with 0.5 mL DI | | | | |
| Tubes filled with | — | — | — | — | — | — | — | — | — | — | — | — | — | — | — | — | — | — | — | — | 1 | 1 | 1 | 1 | 1 |
| minimum expected | | | | | | | | | | | | | | | | | | | | | | | | | |
| dead volume | | | | | | | | | | | | | | | | | | | | | | | | | |

Key
— No Test Performed on tube
0 Failed to pour
1 Full Pour

### B. Tube Pierce & Aspirate Test

A script was written to best mimic the normal operation of the cleaning cycle of the automated analyzer 10 while also minimizing the time to run a large number of pierce and aspirate cycles. Tables 4 and 5 represent testing using the conventional test tube 14 and the striated tube 28, respectively. The test pierced and aspirates each tube 14, 28 a total of 80 times. The 80 pierces of each tube 14, 28 are divided into four rounds, each consisting of 20 aspiration cycles. The intent of the rounds was to allow time after 20 aspirations to manually remove the cap and replace the cap using a cap torqueing tool to a design specified 6 in-lbs. This was done to allow the internal pressure to equalize to atmosphere in the case that the rate at which the aspirations were being performed may cause a larger vacuum than normal operation in the tubes 14, 28, thus affecting the results. This has the potential to impact results though in that the tube is being handled after every 20 pierces, which is not part of normal operation as the users would likely never remove the cap.

TABLE 4
Conventional tube 14 instrument pierce baseline testing results
Current Tube, Ventana Filled, Non-Expired

| Pierce # | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Tube 1 | | | | | | | | | | | | | | | | | | | | |
| Round 1 | 1 | 1 | 1 | 1 | 0 | 1 | 1 | 1 | 1 | 1 | 1 | 1 | 1 | 1 | 1 | 1 | 1 | 1 | 1 | 1 |
| Round 2 | 1 | 1 | 1 | 1 | 1 | 1 | 1 | 1 | 1 | 1 | 1 | 1 | 1 | 1 | 1 | 1 | 1 | 1 | 1 | 1 |
| Round 3 | 1 | 1 | 1 | 1 | 1 | 1 | 1 | 1 | 1 | 1 | 1 | 1 | 1 | 1 | 1 | 1 | 1 | 1 | 1 | 1 |
| Round 4 | 1 | 1 | 1 | 1 | 1 | 1 | 1 | 1 | 1 | 1 | 1 | 1 | 1 | 1 | 1 | 1 | 1 | 1 | 1 | 1 |
| Tube 2 | | | | | | | | | | | | | | | | | | | | |
| Round 1 | 1 | 1 | 1 | 1 | 0 | 0 | 0 | 0 | 0 | 0 | 0 | 0 | 0 | 0 | 0 | 0 | 0 | 0 | 0 | 0 |
| Round 2 | 1 | 1 | 1 | 1 | 0 | 0 | 1 | 1 | 1 | 1 | 1 | 1 | 1 | 1 | 1 | 1 | 1 | 1 | 1 | 1 |
| Round 3 | 0 | 0 | 0 | 0 | 0 | 0 | 0 | 0 | 0 | 0 | 0 | 0 | 1 | 1 | 1 | 1 | 1 | 1 | 1 | 1 |
| Round 4 | 1 | 1 | 1 | 1 | 1 | 1 | 1 | 1 | 1 | 1 | 1 | 1 | 1 | 1 | 1 | 1 | 1 | 1 | 1 | 1 |
| Tube 3 | | | | | | | | | | | | | | | | | | | | |
| Round 1 | 1 | 1 | 1 | 1 | 0 | 0 | 0 | 0 | 0 | 0 | 0 | 0 | 0 | 0 | 0 | 0 | 0 | 0 | 0 | 0 |
| Round 2 | 0 | 1 | 1 | 1 | 1 | 1 | 1 | 1 | 1 | 1 | 1 | 1 | 1 | 1 | 1 | 1 | 1 | 1 | 1 | 1 |
| Round 3 | 1 | 1 | 1 | 1 | 1 | 1 | 1 | 1 | 1 | 1 | 1 | 1 | 1 | 1 | 1 | 1 | 1 | 1 | 1 | 1 |
| Round 4 | 1 | 1 | 1 | 1 | 1 | 1 | 1 | 1 | 1 | 1 | 1 | 1 | 1 | 1 | 1 | 1 | 1 | 1 | 1 | 1 |
| Tube 4 | No Vent | | | | | | | | | | | | | | | | | | | |
| Round 1 | 1 | 1 | 1 | 1 | 0 | 1 | 1 | 1 | 1 | 1 | 1 | 1 | 1 | 1 | 1 | 1 | 1 | 1 | 1 | 1 |
| Round 2 | 1 | 1 | 1 | 1 | 1 | 1 | 1 | 1 | 1 | 1 | 1 | 1 | 1 | 1 | 1 | 1 | 1 | 1 | 1 | 1 |
| Round 3 | 1 | 1 | 1 | 1 | 1 | 1 | 1 | 1 | 1 | 1 | 1 | 1 | 0 | 1 | 1 | 1 | 0 | 1 | 1 | 1 |
| Round 4 | 0 | 1 | 1 | 1 | 0 | 0 | 0 | 0 | 0 | 0 | 0 | 0 | 0 | 0 | 0 | 0 | 0 | 0 | 0 | 0 |
| | Vent Tube | | | | | | | | | | | | | | | | | | | |
| Round 5 | 0 | 0 | 0 | 0 | 0 | 0 | 0 | 0 | | | | | | | | | | | | |
| Tube 5 | No Vent | | | | | | | | | | | | | | | | | | | |
| Round 1 | 1 | 1 | 1 | 1 | 0 | 0 | 0 | 1 | 1 | 1 | 1 | 1 | 0 | 1 | 1 | 1 | 1 | 1 | 1 | 1 |
| Round 2 | 1 | 1 | 1 | 1 | 1 | 1 | 1 | 1 | 1 | 1 | 1 | 1 | 1 | 1 | 1 | 1 | 1 | 1 | 1 | 1 |
| Round 3 | 1 | 1 | 1 | 1 | 1 | 1 | 1 | 1 | 1 | 1 | 1 | 1 | 1 | 1 | 1 | 1 | 1 | 1 | 1 | 1 |
| Round 4 | 1 | 1 | 1 | 1 | 1 | 1 | 1 | 1 | 1 | 1 | 1 | 1 | 1 | 1 | 1 | 1 | 1 | 1 | 1 | 1 |

TABLE 5
Convex Striated tube 28 (FIG. 5) instrument pierce testing results. Yellow highlighting represents
where observed changes in the sample cup were likely due to dispensing air, and thus,
the exact pierce number is unknown.
Protomold Striation Tube, Decanted, Non-Explred

| Pierce # | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| Tube 1 | Vented at beginning of each round starting round 2 | | | | | | | | | | | | | | | | | | | |
| Round 1 | 1 | 1 | 1 | 1 | 1 | 1 | 1 | 1 | 1 | 1 | 1 | 1 | 1 | 1 | 1 | 1 | 1 | 1 | 1 | 1 |
| Round 2 | 1 | 1 | 1 | 1 | 1 | 1 | 1 | 1 | 1 | 1 | 1 | 1 | 1 | 1 | 1 | 1 | 1 | 1 | 1 | 1 |
| Round 3 | 1 | 1 | 1 | 1 | 1 | 1 | 1 | 1 | 1 | 1 | 1 | 1 | 1 | 1 | 1 | 1 | 1 | 1 | 1 | 1 |
| Round 4 | 1 | 1 | 1 | 1 | 1 | 1 | 1 | 1 | 0 | 0 | 0 | 0 | 0 | 0 | 0 | 0 | 0 | 0 | 0 | 0 |
| Tube 2 | non-vented | | | | | | | | | | | | | | | | | | | |
| Round 1 | 1 | 1 | 1 | 1 | 1 | 1 | 1 | 1 | 1 | 1 | 1 | 1 | 1 | 1 | 1 | 1 | 1 | 1 | 1 | 1 |
| Round 2 | 1 | 1 | 1 | 1 | 1 | 1 | 1 | 1 | 1 | 1 | 1 | 1 | 1 | 1 | 1 | 1 | 1 | 1 | 1 | 1 |
| Round 3 | 1 | 1 | 1 | 1 | 1 | 1 | 1 | 1 | 1 | 1 | 1 | 1 | 1 | 1 | 1 | 1 | 1 | 1 | 1 | 1 |
| Round 4 | 1 | 1 | 1 | 1 | 1 | 1 | 1 | 1 | 1 | 1 | 1 | 0 | 0 | 0 | 0 | 0 | 0 | 0 | 0 | 0 |
| Tube 3 | non-vented | | | | | | | | | | | | | | | | | | | |
| Round 1 | 1 | 1 | 1 | 1 | 1 | 1 | 1 | 1 | 1 | 1 | 1 | 1 | 1 | 1 | 1 | 1 | 1 | 1 | 1 | 1 |
| Round 2 | 1 | 1 | 1 | 1 | 1 | 1 | 1 | 1 | 1 | 1 | 1 | 1 | 1 | 1 | 1 | 1 | 1 | 1 | 1 | 1 |
| Round 3 | 1 | 1 | 1 | 1 | 1 | 1 | 1 | 1 | 1 | 1 | 1 | 1 | 1 | 1 | 1 | 1 | 1 | 1 | 1 | 1 |
| Round 4 | 1 | 1 | 1 | 1 | 1 | 1 | 1 | 1 | 1 | 1 | 0 | 0 | 0 | 0 | 0 | 0 | 0 | 0 | 0 | 0 |
| Tube 4 | | | | | | | | | | | | | | | | | | | | |
| Round 1 | 1 | 1 | 1 | 1 | 1 | 1 | 1 | 1 | 1 | 1 | 1 | 1 | 1 | 1 | 1 | 1 | 1 | 1 | 1 | 1 |
| Round 2 | 1 | 1 | 1 | 1 | 1 | 1 | 1 | 1 | 1 | 1 | 1 | 1 | 1 | 1 | 1 | 1 | 1 | 1 | 1 | 1 |
| Round 3 | 1 | 1 | 1 | 1 | 1 | 1 | 1 | 1 | 1 | 1 | 1 | 1 | 1 | 1 | 1 | 1 | 1 | 1 | 1 | 1 |
| Round 4 | 1 | 1 | 1 | 1 | 1 | 1 | 1 | 1 | 0 | 0 | 0 | 0 | 0 | 0 | 0 | 0 | 0 | 0 | 0 | 0 |

c. The various striated embodiments may be applicable to reducing flow losses in extruded tubing for flow.

Accordingly, by the above disclosure, in one aspect a fluid-holding vessel with a surface tension reducing inner surface striated geometry is disclosed and described which addresses the above noted issues. The fluid-holding vessel may be a test tube that is used in combination with a cap, which is penetrable by a fluid transfer device of an automated analyzer used to transfer fluids to or from the striated test tube, where the tube and cap may remain physically and sealably associated during a fluid transfer. The automated analyzer may be used in combination with the fluid-holding vessel as disclosed and described herein, in which the striated geometry of the vessel addresses an aspiration problem of a cleaning fluid dispensed therefrom automatically by the automated analyzer into a sample cup.

In another aspect, a fluid transfer method in which a fluid is drawn from the fluid-holding vessel disclosed and described above via a fluid transfer device of an automated analyzer penetrating a cap physically and sealably associated with the vessel during a fluid transfer, wherein the surface tension reducing inner surface striated geometry of the vessel addresses aspiration problems of a cleaning fluid contained therein such that the cleaning fluid is dispensed by the fluid transfer device into a sample cup. Other more specific embodiments are further disclosed hereinafter.

### Embodiment 1

A fluid-holding vessel (28) with a surface tension reducing inner surface striated geometry that permits a liquid (18) when contained therein to freely flow from the vessel under the force of gravity, wherein said geometry comprises longitudinally extending striations (24) provided spaced from each other along an interior inner diameter (ID) of an interior surface (26) of the vessel (28), each striation (24) has a macroscopic profile, either proud or recessed to the interior surface (26) of the vessel (28), which aids in breaking surface tension, thereby lowering surface forces between the liquid (18) and the interior surface (26) of the fluid-holding vessel (28).

### Embodiment 2

The fluid-holding vessel (28) according to Embodiment 1, wherein the vessel (28) has a bottom (32), an opening 34 opposed to the bottom (32), and a sidewall (30) that is integrally formed at least with the striations (24) and the interior surface (26).

### Embodiment 3

The fluid-holding vessel (28) according to Embodiment 2, wherein the bottom (32) has a shape that is curved, flat, sloped, concave, convex or any other suitably shaped bottom.

### Embodiment 4

The fluid-holding vessel (28) according to Embodiment 2, wherein the sidewall (30) is inserted into a tube (14).

### Embodiment 5

The fluid-holding vessel (28) according to Embodiment 2, wherein thickness of the sidewall (30) is constant from the bottom (32) to the opening (34).

### Embodiment 6

The fluid-holding vessel (28) according to Embodiment 2, wherein thickness of the sidewall (30) tapers from the bottom (32) to the opening (34).

### Embodiment 7

The fluid-holding vessel (28) according to Embodiment 6, wherein the taper of the sidewall (30) is a continuous taper from the bottom (32) to the opening (34).

### Embodiment 8

The fluid-holding vessel (28) according to Embodiment 7, wherein the continuous taper ranges from 0.4° to 3°, and is preferably 2°.

### Embodiment 9

The fluid-holding vessel (28) according to Embodiment 1, wherein the taper varies in draft along length (L) of the vessel (28).

### Embodiment 10

The fluid-holding vessel (28) according to Embodiment 9, wherein the interior surface (26) has a first taper for a first portion A that extends from a bottom (32), a second portion B with a second taper, the second portion being adjacent the first portion A and the second taper being greater than the first taper, and a third portion C comprising a remainder of the length L of the vessel (28) to an opening (34) that is opposite to the bottom (32) and provided with a third taper, the third taper being greater than the second taper.

### Embodiment 11

The fluid-holding vessel (28) according to Embodiment 10, wherein the first portion A ranges in length from 0.5 to 1.5 inches (1.27 cm to 3.81 cm) from the bottom (32), the second portion B from 0.5 to 1.5 inches (1.27 cm to 3.81 cm), and in a preferred embodiment portions A and B are each 1 inch (2.54 cm) in length.

### Embodiment 12

The fluid-holding vessel (28) according to Embodiment 10, wherein the first taper is 0.5° of taper, the second taper is 1° in taper, and third taper is 2° of taper.

### Embodiment 13

The fluid-holding vessel (28) according to any one of the previous Embodiments 1-12, wherein the macroscopic profile of each striation (24) is either convex or concave, and each striation (24) has either the same or a different macroscopic profile from other ones of the striations (24).

### Embodiment 14

The fluid-holding vessel (28) according to any one of the previous Embodiments 1-13, wherein each striation (24) is provided along an interior inner diameter (ID) of the interior surface (26) parallel to a longitudinal axis (X) of the fluid-holding vessel (28).

### Embodiment 15

The fluid-holding vessel (28) according to any one of the previous Embodiments 1-14, wherein the striations (24) range from 4 to 24 in number, and preferably 8 to 12 in number.

### Embodiment 16

The fluid-holding vessel (28) according to any one of the previous Embodiments 1-15, wherein the striations (24) are spaced equally or unequally from each other, and have the same or alternating patterns of striations (24) of different shapes, the different shapes being wider and/or narrow valleys in the case of concave striations, higher and/or short hills in the case of convex striations, and combinations thereof.

### Embodiment 17

The fluid-holding vessel (28) according to any one of the previous Embodiments 1-16, wherein at least the striations (24) are constructed from a material selected from polymeric materials, polystyrene, polypropylene, polycarbonate, polyvinylchloride, polytetra-fluoroethylene, or other suitable polyolefin.

### Embodiment 18

The fluid-holding vessel (28) according to any one of the previous Embodiments 1-17, wherein the fluid-holding vessel (28) has an interior volume which ranges from 2 ml to 40 ml.

### Embodiment 19

The fluid-holding vessel (28) according to Embodiment 1, wherein the vessel (28) is a cylindrical tube that has a length L ranging from 7 to 8 cm, an outside diameter of 1 to 2 cm and provided with threads, an internal draft that ranges from 0.4 to 0.6 degrees, wherein the striations (24) total twelve concave striations that are space equally from each other, and a cross section of each striation (24) is identical to each other and has a depth that ranges from 0.5 to 0.6 mm below the interior surface (26) with a minor radius that ranges from 0.3 to 0.4 mm and a major radius that ranges from 3 to 4 mm, wherein a minor internal diameter that is adjacent a bottom (32) of the vessel (28) ranges from 0.7 to 0.8 cm, and a major internal diameter that is adjacent an opening (34) of the vessel (28) ranges from 0.8 to 0.9 cm.

### Embodiment 20

The fluid-holding vessel (28) according to any one of the previous Embodiments 1-19, wherein the interior surface (26, 44) of the vessel (28) is fluorinated.

### Embodiment 21

The fluid-holding vessel (28) according to any one of the previous Embodiments 1-20, wherein the fluid-holding vessel 28 has a shape selected from round, triangular, square and other multisided tubing.

### Embodiment 22

The fluid-holding vessel (28) according to any one of the previous Embodiments 1-21 in combination with a cap (20) which is penetrable by a fluid transfer device (11) of an automated analyzer (10) used to transfer fluids to or from the vessel (28), wherein the vessel (28) and cap (20) remain physically and sealably associated during a fluid transfer.

### Embodiment 23

The fluid-holding vessel (28) according to any one of the previous Embodiments 1-22 in combination with an automated analyzer (10), wherein the automated analyzer (10) is configured to aspirate a cleaning fluid from the vessel (28).

### Embodiment 24

A fluid transfer method in which a fluid is drawn from a fluid-holding vessel (28) according to any one of the previous Embodiments 1-22 via a fluid transfer device (11) of an automated analyzer (10).

### Embodiment 25

The fluid-holding vessel (28) according to any one of the previous embodiments 1-24 in which the fluid (18) is water, a cleaning fluid, a bleach solution, a hypochlorite based disinfectant solution, a sodium hypochlorite based disinfectant solution, or a 0.7% sodium hypochlorite based disinfectant solution.

While various embodiments herein have been described and shown in considerable detail with reference to certain preferred embodiments, those skilled in the art will readily appreciate other embodiments of the present invention. Accordingly, the present invention is deemed to include all modifications and variations encompassed within the spirit and scope of the following appended claims.

1. A fluid-holding vessel (28) with a surface tension reducing inner surface striated geometry that permits a liquid (18) when contained therein to freely flow from the vessel under the force of gravity, wherein said geometry comprises longitudinally extending striations (24) provided spaced from each other along an interior surface (26) of the vessel (28), each striation (24) has a macroscopic profile, either proud or recessed to the interior surface (26) of the vessel (28), which aids in breaking surface tension, thereby lowering surface forces between the liquid (18) and the interior surface (26) of the fluid-holding vessel (28).

2. The fluid-holding vessel (28) according to claim 1, wherein the vessel (28) has a bottom (32), an opening 34 opposed to the bottom (32), and a sidewall (30) that is integrally formed at least with the striations (24) and the interior surface (26).

3. The fluid-holding vessel (28) according to claim 2, wherein the bottom (32) has a shape that is curved, flat, sloped, concave, convex or any other suitably shaped bottom.

4. The fluid-holding vessel (28) according to claim 2, wherein the sidewall (30) is inserted into a tube (14).

5. The fluid-holding vessel (28) according to claim 2, wherein thickness of the sidewall (30) is constant from the bottom (32) to the opening (34).

6. The fluid-holding vessel (28) according to claim 2, wherein thickness of the sidewall (30) tapers from the bottom (32) to the opening (34).

7. The fluid-holding vessel (28) according to claim 6, wherein the taper of the sidewall (30) is a continuous taper from the bottom (32) to the opening (34).

8. The fluid-holding vessel (28) according to claim 7, wherein the continuous taper ranges from 0.4° to 3°, and is preferably 2°.

9. The fluid-holding vessel (28) according to claim 1, wherein the taper varies in draft along length (L) of the vessel (28).

10. The fluid-holding vessel (28) according to claim 9, wherein the interior surface (26) has a first taper for a first portion A that extends from a bottom (32), a second portion B with a second taper, the second portion being adjacent the first portion A and the second taper being greater than the first taper, and a third portion C comprising a remainder of the length L of the vessel (28) to an opening (34) that is opposite to the bottom (32) and provided with a third taper, the third taper being greater than the second taper.

11. The fluid-holding vessel (28) according to claim 10, wherein the first portion A ranges in length from 0.5 to 1.5 inches (1.27 cm to 3.81 cm) from the bottom (32), the second portion B from 0.5 to 1.5 inches (1.27 cm to 3.81 cm), and in a preferred embodiment portions A and B are each 1 inch (2.54 cm) in length.

12. The fluid-holding vessel (28) according to claim 10, wherein the first taper is 0.5° of taper, the second taper is 1° in taper, and third taper is 2° of taper.

13. The fluid-holding vessel (28) according to claim 1, wherein the macroscopic profile of each striation (24) is either convex or concave, and each striation (24) has either the same or a different macroscopic profile from other ones of the striations (24).

14. The fluid-holding vessel (28) according to claim 1, wherein each striation (24) is provided along an interior inner diameter (ID) of the interior surface (26) parallel to a longitudinal axis (X) of the fluid-holding vessel (28).

15. The fluid-holding vessel (28) according to claim 1, wherein the striations (24) range from 4 to 24 in number, and preferably 8 to 12 in number.

16. The fluid-holding vessel (28) according to claim 1, wherein the striations (24) are spaced equally or unequally from each other, and have the same or alternating patterns of striations (24) of different shapes, the different shapes being wider and/or narrow valleys in the case of concave striations, higher and/or short hills in the case of convex striations, and combinations thereof.

17. The fluid-holding vessel (28) according to claim 1, wherein at least the striations (24) are constructed from a material selected from polymeric materials, polystyrene, polypropylene, polycarbonate, polyvinylchloride, polytetra-fluoroethylene, or other suitable polyolefin.

18. The fluid-holding vessel (28) according to claim 1, wherein the fluid-holding vessel (28) has an interior volume which ranges from 2 ml to 40 ml.

19. The fluid-holding vessel (28) according to claim 1, wherein the vessel (28) is a cylindrical tube that has a length L ranging from 7 to 8 cm, an outside diameter of 1 to 2 cm and provided with threads, an internal draft that ranges from 0.4 to 0.6 degrees, wherein the striations (24) total twelve concave striations that are space equally from each other, and a cross section of each striation (24) is identical to each other and has a depth that ranges from 0.5 to 0.6 mm below the interior surface (26) with a minor radius that ranges from 0.3 to 0.4 mm and a major radius that ranges from 3 to 4 mm, wherein a minor internal diameter that is adjacent a bottom (32) of the vessel (28) ranges from 0.7 to 0.8 cm, and a major internal diameter that is adjacent an opening (34) of the vessel (28) ranges from 0.8 to 0.9 cm.

20. The fluid-holding vessel (28) according to claim 1, wherein the interior surface (26, 44) of the vessel (28) is fluorinated.

21. The fluid-holding vessel (28) according to claim 1, wherein the fluid-holding vessel 28 has a shape selected from round, triangular, square and other multisided tubing.

22. The fluid-holding vessel (28) according to claim 1 in combination with a cap (20) which is penetrable by a fluid transfer device (11) of an automated analyzer (10) used to transfer fluids to or from the vessel (28), wherein the vessel (28) and cap (20) remain physically and sealably associated during a fluid transfer.

23. The fluid-holding vessel (28) according to claim 1 in combination with an automated analyzer (10), wherein the automated analyzer (10) is configured to aspirate a cleaning fluid from the vessel (28).

24. A fluid transfer method in which a fluid is drawn from a fluid-holding vessel (28) according to claim 1 via a fluid transfer device (11) of an automated analyzer (10).

25. The fluid-holding vessel (28) according to claim 1 in which the fluid (18) is water, a cleaning fluid, a bleach solution, a hypochlorite based disinfectant solution, a sodium hypochlorite based disinfectant solution, or a 0.7% sodium hypochlorite based disinfectant solution.
